(19) United States
(12) Patent Application Publication
(10) Pub. No.: US 2014/0328945 A1
(43) Pub. Date: Nov. 6, 2014

(54) METHOD FOR STABILIZING AN ELECTROCHEMICALLY GENERATED SANITIZING SOLUTION HAVING A PREDETERMINED LEVEL OF FREE AVAILABLE CHLORINE AND pH

(71) Applicant: Aquaxo, Inc., Fontana, CA (US)

(72) Inventors: Phillip Adams, Roswell, GA (US); Michel van Schaik, Loxahatchee, FL (US)

(73) Assignee: Aquaxo, Inc., Fontana, CA (US)

(21) Appl. No.: 13/887,147

(22) Filed: May 3, 2013

(51) Int. Cl. A01N 59/00; A61K 33/20; A23L 3/358

(52) U.S. Cl. CPC A01N 59/00; A23L 3/358; A61K 33/20; A23V 2002/00
USPC 424/661; 426/335

(57) ABSTRACT

The present invention provides a method for stabilizing free available chlorine solutions that are electrochemically generated utilizing one or more Cylindrical Electrolysis cells, which allows generation of Hypochlorous Acid (HOCL) solutions with excellent sanitizing properties. The invention further provides methods to stabilize different concentrations of Hypochlorous Acid solutions with a pH value ranging from 4.0 to 7.5 and an Redox Oxidation Potential between +700 and +1200 mV, as well methods to stabilize hydrogel formulations containing Hypochlorous Acid as the active ingredient.

### REFERENCE TO RELATED APPLICATIONS

The present invention is related to U.S. patent application Ser. No. 13/718,677, filed Dec. 18, 2012, and entitled “APPARATUS AND METHOD FOR GENERATING A STABILIZED SANITIZING SOLUTION”; U.S. patent application Ser. No. 13/718,721, filed Dec. 18, 2012, and entitled “MESH ELECTRODE ELECTROLYSIS APPARATUS AND METHOD FOR GENERATING A SANITIZING SOLUTION”; and U.S. patent application Ser. No. 13/324,714, filed Dec. 13, 2011, entitled “DUAL DIAPHRAGM ELECTROLYSIS CELL ASSEMBLY AND METHOD FOR GENERATING A CLEANING SOLUTION WITHOUT ANY SALT RESIDUES AND SIMULTANEOUSLY GENERATING A SANITIZING SOLUTION HAVING A PREDETERMINED LEVEL OF AVAILABLE FREE CHLORINE AND pH”. The contents of the above referenced applications are incorporated herein by reference in its entirety.

### FIELD OF THE INVENTION

The present invention relates to the field of producing Hypochlorous Acid stabilized solutions and hydrogel formulations of Hypochlorous Acid (HOCl), as well as methods for their production and use. The solution finds use for cleaning, sanitizing and/or disinfecting surfaces, food such as fruit, vegetables and crops, or mammalian tissues (including wounds). The solutions further find use in the preservation of fruit, produce, cut flowers and other agricultural products.

### BACKGROUND OF THE INVENTION

Hypochlorous Acid is an oxidant and biocide that is produced by the human body's natural immune system to fight infection. Hypochlorous Acid is generated as the final step of the Oxidative Burst Pathway, with large quantities of Hypochlorous Acid being released into the phagocytic vesicles to destroy the invading microorganisms. It is considered that Hypochlorous Acid exerts its biocidal effect by attacking the surface and plasma membrane proteins, impairing transport of solutes and the salt balance of bacterial cells (Pieterson et al., Water SA, 22(1):43-48 (1996)). Escherichia coli exposed to Hypochlorous Acid lose viability in less than 100 ms due to inactivation of many vital systems. (Fair et al., 40 J. Am. Water Works Assoc. 1051-61 (1940)). Hypochlorous acid at 2.6 ppm caused 100% growth inhibition of E. coli in dilute bacterial suspensions in about 5 minutes. (Chesney et al., 178 J. Bacteria 2131-2135 (1996)). According to Chemistry of Water Treatment (2^{nd} Edition), S. D. Faust and O. M. Aly (1998), 100% kill in 5 minutes requires only 0.08 ppm for A. aerogenes, 0.06 ppm for S. typhosa, 0.05 ppm for S. clysvnteriae, and 0.03 ppm for E. coli.

Although Hypochlorous Acid is biocidal for microorganisms, it is not significantly toxic to human or animal cells, at least partly because human and animal cells have extensive, highly effective defense mechanisms known as the Antioxidant Defense System (ADS). Hypochlorous Acid has a wide range of applications where it is important to control microbial contamination, such as for the care and management of wounds, disinfecting hard surfaces such as medical or dental equipment, food safety and processing, water treatment, as well as other industrial and agricultural applications.

One limitation associated with Hypochlorous Acid solutions is their stability, which has limited much of the commercial use to those situations where the solution can be made on site for relatively immediate use. Existing alternatives include Dakin's solution for wound care, which is a diluted sodium hypochlorite solution (0.5%) prepared by mixing sodium hypochlorite (5.25%), sodium bicarbonate/carbonate (1%), and clean tap water. However, Dakin's solution has a high pH, and thus causes pain and burning in wound treatment along with rashes, itching, swelling, hives, and/or blisters. Further, Dakin's solution is unstable and unsuited for clinical use at lower pH's (<8.5). Another alternative is the Microcyn™ solution. While Microcyn™ has a 2 year shelf life, it suffers from a limited initial level of Free Available Chlorine (FAC) of about 80 parts per million and at a pH of 7.4, a lower percent of Hypochlorous Acid, which may limit its biocidal effectiveness. The Microcyn™ solution has a conductivity of between 1500 and 2000 uS and has an osmolarity of about 10 to 50 osmoles per liter. Another alternative is Vashe™: Vashe™ has a 2 year shelf life and contains about 240 parts per million FAC. The Vashe™ solution is relatively stable in terms of FAC, but its pH, starting as pH 5.8 decreases significantly to approximately pH 3.3 causing a burning effect when applied as wound treatment. Further, the Vashe™ solution consist of a much higher conductivity of about 6,000 to 7,000 uS, which increases the osmolarity to far above 100 osmoles per liter. EcaFlo™ is available only for hard surface disinfection. This solution contains equimolar amounts of hypochlorite and Hypochlorous Acid in addition to a high sodium chloride content. Conductivity of EcaFlo™ is about 12,000 uS to 16,000 uS, which limit its usage a hard surface disinfectant due to leaving salt residues on the surface. Due to the high conductivity and osmolarity EcaFlo™ has no usages in wound treatment. The pH of the solution is around 7.5 and the solution has an FAC content of approximately 460 ppm. The solution has a relatively short shelf life of 30 days.

There is an unmet need for a Hypochlorous Acid solution that has a high FAC content (≧200 ppm), a stable neutral pH (5-7), a low conductivity (≦2000 uS), a low salinity (0.05% wt), a low osmolarity (50 Osm/l) and has stability properties required to be commercially useful in medical and other commercial settings, and is not irritating or harmful to humans. The claimed invention meets these and other toxicological and microbial objectives.

### SUMMARY OF THE INVENTION

The present invention provides a stabilized Hypochlorous Acid solution or hydrogel formulation thereof, which may be conveniently packaged for sale, or stored for later use on demand. The invention further provides methods of making the stabilized Hypochlorous Acid solution or hydrogel formulation thereof, as well as methods of use for disinfecting mammalian tissue, including wounds and burns, disinfecting or cleansing hard surfaces, treating (e.g., preserving and/or disinfecting) food products or cut flowers, among other uses.

In one aspect, the invention provides a stabilized Hypochlorous Acid solution. The solution incorporates a stabilizing amount of dissolved ionic compounds (DIC), which can be in the form of a sodium phosphate, sodium polyphosphate or a phosphate or polyphosphate of an alkali or alkaline earth metal. The solution may have a Free Available Chlorine (FAC) content of from about 10 to about 1000 parts per million, and a pH of from about 4.0 to about 7.5. For example, in certain embodiments, the solution has a pH of from about 5 to about 6. In certain embodiments, the solution contains Hypochlorous Acid, and is prepared by electrolysis of a brine solution. The solution is stabilized, as determined by its change in pH and/or FAC over time, for at least six months, but in various embodiments, the solution is stabilized for at least twelve months, or more.

In certain embodiments, sodium phosphate, sodium polyphosphate or a blended DIC solution is incorporated into the solution or hydrogel formulation at a level of about 1:10 to about 1:1000 molar ratio relative to the FAC content. For example, a blended DIC solution may be added at a level of about 1:5000, about 1:1000, about 1:500 or about 1:100 or at a larger (i.e., less dilute) molar ratio relative to the FAC content. In certain embodiment, a blended DIC solution is incorporated into the solution at a level of about 1:250 relative to the FAC content. While the solution may contain other buffers in some embodiments, in other embodiments, the solution does not contain, or contains only limited, a sodium phosphate, sodium polyphosphate or blended DIC solution as buffer. For example, the solution may comprise Hypochlorous Acid produced by electrolysis of a brine solution, and the solution may have an FAC content of from about 10 to about 1000 parts per million, a pH in the range of about 4 to about 7.5, a conductivity of about 100 to about 15,000 uS, and an amount of dissolved ionic compounds (DIC) in the range of about 1 to about 100 parts per million. In some embodiments, the conductivity of the solution does not impact the amount of DIC needed for solution stabilization. In certain embodiments, the Hypochlorous Acid solution is formulated as a hydrogel.

In another aspect, the invention provides a method for preparing the stabilized Hypochlorous Acid solution. The method involves incorporating the DIC (e.g., in the form of a sodium phosphate polyphosphate or blend of phosphates) by addition to an electrolyte for electrochemical treatment, or incorporating the DIC (e.g., in the form of phosphate, polyphosphate or a blend of phosphates) by directly adding to an electrochemically generated Hypochlorous Acid solution.

In some embodiments, the method involves incorporation of an electrochemically generated buffer (e.g., in the form of Hydroxide) by addition to an electrolyte for electrochemical treatment or incorporating the electrochemically generated buffer to an electrochemically generate Hypochlorous Acid solution.

Still other objectives of the invention provides methods of disinfecting, cleansing, or treating a mammalian tissue, such as a wound, burn, or dermatosis, or provides methods of sanitizing, disinfecting or cleansing a hard surface, or provides methods for treating or preserving a food or agricultural product or cut flowers. Due to the stability of the Hypochlorous Acid solutions and Hydrogel formulations, such methods need not be performed proximately to the production of the biocidal solution. Further, as shown herein, stabilized Hypochlorous Acid solutions or hydrogel formulations of the invention maintain activity even in the presence of high organic load. In still other embodiments, the invention provides a method for treating a skin condition, including dermatosis, rosasea, skin infection, skin allergy, psoriasis, or acne. In such embodiments, the HOCl may be formulated as a hydrogel.

Other objectives and further advantages and benefits associated with this invention will be apparent to those skilled in the art.

### BRIEF DESCRIPTION OF THE FIGURES

FIG. 1 is a chart of a first test to depict the change in pH and FAC over time of a stabilized electrochemically HOCl solution in an unsealed HDPE bottle;

FIG. 2 is a chart of a second test to depict the change in pH and FAC over time of a stabilized electrochemically HOCl solution in an unsealed HDPE bottle;

FIG. 3 is a chart of a first test that depicts the change in pH and FAC over time of an electrochemically generated HOCl solution stored in unsealed HDPE bottle;

FIG. 4 is a chart of a second test that depicts the change in pH and FAC over time of an electrochemically generated HOCl solution stored in unsealed HDPE bottle;

FIGS. 5 and 5A is a chart that shows the change in pH and FAC over time of a stabilized electrochemically generated HOCl solution (buffered) stored in sealed Jerri cans, drums and totes at ambient temperature;

FIG. 6 is a chart that depicts the change in in pH and FAC over time of an electrochemically generated HOCl solution (non-buffered) stored sealed Jerri cans, drums and totes at ambient temperature;

FIG. 7 is a chart that depicts the efficacy of a stabilized electrochemically HOCl solution (buffered);

FIG. 8 is a chart that depicts the efficacy of an electrochemically generated HOCL solution (non-buffered);

FIG. 9 is a chart that depicts the toxicity of a stabilized electrochemically HOCl solution (buffered);

FIG. 10 is a chart that depicts the toxicity of an electrochemically generated HOCl solution (non-buffered);

FIG. 11 is a chart and graphs that rates the extended stability of samples of batches stabilized (buffered) electrochemically generated Hypochlorous Acid solutions;

FIG. 12 is a chart to depict the effect of electrochemically generated Hydroxide addition in the electrolyte on pH and solution stability in the electrochemically generated HOCL solution—Amperage vs. FAC (Sodium Chloride Brine pH=7.0);

FIG. 13 is a further chart to depict the effect of electrochemically generated Hydroxide addition in the electrolyte on pH and solution stability in the electrochemically generated HOCL solution—Amperage vs. FAC (Sodium Chloride Brine pH=9.0);

FIG. 14 is a chart to depict a shift in pH upon formulation as a hydrogel; and

FIG. 15 is a chart to illustrate the resultant FAC and pH as various concentrations of polyacrylate are added.

### DETAILED DESCRIPTION OF THE PREFERRED EMBODIMENT

The present invention provides a stabilized Hypochlorous Acid solution or hydrogel formulation thereof, which may be conveniently packaged for sale, or stored for later use on demand. The invention further provides methods of making the stabilized Hypochlorous Acid solution, as well as methods of use for disinfecting mammalian tissue, including wounds and burns, disinfecting or cleansing surfaces, or treating or preserving food products or cut flowers, among other uses.

In one aspect, the invention provides a stabilized Hypochlorous Acid solution or hydrogel formulation thereof. The solution incorporates a stabilizing amount of dissolved ionic compounds (DIC), such as a sodium phosphate, sodium polyphosphates or a blended phosphate or polyphosphate solution of an alkali or alkaline earth metal. The solution may have a Free Available Chlorine (FAC) content of from about 10 to about 1000 parts per million, and a pH of from about 4.0 to about 7.5. In certain embodiments, the solution's active ingredient is Hypochlorous Acid, and is prepared by electrolysis of a brine solution. The solution is stabilized, as determined by its change in pH and/or FAC over time, for at least six months, but in various embodiments, the solution is stabilized for at least one year, or more.

The Hypochlorous Acid solution may be generated by electrolysis of a brine solution, such as sodium or potassium chloride, and comprise a mixture of oxidizing species such as predominantly Hypochlorous Acid and sodium hypochlorite. Hypochlorous Acid and hypochlorite are in equilibrium and the position of the equilibrium is determined predominately by the pH (that is, pH effects the concentration of each component). An electrolyzed sodium chloride solution with a pH of 5.1 to 6.0 has a purity of about ≧95% Hypochlorous Acid. Thus, the electrolyzed solution supplied may have a pH of from about 4.0 to about 7.5, but in certain embodiments has a pH of from about 4 to about 5, or a pH of about 5 to about 6, or a pH of from about 5.5 to about 6.5, or a pH of from about 6 to about 7. At a pH of about 5.4 the solution will contain mostly (close to 100%) Hypochlorous Acid with respect to hypochlorite.

While the solution may comprise, or consist essentially of Hypochlorous Acid as the active agent, in some embodiments, it may contain other oxidants. In some embodiments, the solution contains other oxidizing or radical producing species such as hypochlorite, hydroxide, H₂O₂ and O₃, among others.

The biocidal activity of the solution can be expressed in terms of Free Available Chlorine or FAC. While the invention is applicable to an FAC range of from about 10 to about 1000 ppm, in certain embodiments, the solution has a relatively high FAC content and is suitable for use with mammalian tissues or agricultural products. For example, the solution may have an FAC content of from about 10 to 1000 ppm, or 100 to 150 ppm, or 180 to 220, or about 225 to about 250 ppm. Other FAC levels may be employed, and may be selected based upon the intended application. For example, without any limitation, for surface disinfection the FAC may be in the range of about 400 to about 500 ppm, or for disinfection of water about 600 to about 1000 ppm.

While the Hypochlorous Acid may be produced chemically in accordance with some embodiments (e.g., by acidification of hypochlorite), the Hypochlorous Acid may also be produced electrochemically. The electrochemical generation of Hypochlorous Acid is by treatment of a diluted brined solution in one or more cylindrical electrolytic cells. Electrochemical treatment of a brine solution is described, for example, in U.S. Pat. No. 7,303,660, U.S. Pat. No. 7,374,645, U.S. Pat. No. 7,691,249, U.S. Pat. No. 7,828,942, and U.S. Pat. No. 7,897,023, which are hereby incorporated by reference in their entireties.

The solution employs a stabilizing amount of dissolve ionic compounds (DIC), which may be a sodium phosphate, sodium polyphosphate or a blended solution of alkali or alkaline earth metal, such as, for example, sodium, potassium, calcium, or magnesium. In some embodiments, the DIC is added prior to the formation of Hypochlorous Acid (e.g., prior to electrochemical treatment), and in other embodiments, the DIC is added to the solution after electrochemically generation of Hypochlorous Acid. For example, the DIC may be added with or without electrochemically generated Hydroxide to the precursor solution, the electrolyte, and/or the end solution.

The DIC is incorporated at a “stabilizing amount,” which can be determined with reference to the change in the pH or FAC content of the solution over time. Generally, the solution is considered stabilized if the amount of FAC does not drop below about 75% of the initial value over a period of about 6 months. In certain embodiments, the FAC content is stabilized for at least one year from the production date of the solution. Further, the stability of the solution may be determined with reference to the pH. Generally, the solution is considered stabilized if the pH does not vary by 1 unit over a period of about 6 months. In certain embodiments, the pH is stabilized for at least one year from the production date of the solution. The solution should be stored at 25° C. or at 20° C. or less for greater stability. 25° C. and 20° C. are the reference temperatures for determination of stability. For stability testing, solutions are packaged in HDPE bottles (spray cap), ferry cans, drums or totes. Unsealed bottles were stored at 40 degrees Celsius and 75% relative humidity. Solutions stored in ferry cans, drums and totes were kept at ambient temperature and weekly opened for obtaining a sample.

The stabilizing amount of DIC can be determined with reference to the FAC content. For example, in certain embodiments, the stabilizing amount of DIC is incorporated into the solution at a molar ratio of from about 1:250 with respect to the FAC level. In some embodiments, the phosphates and polyphosphates are incorporated into the solution in at least equimolar amounts with respect to the FAC content (e.g., Hypochlorous Acid content). In still other embodiments, the DIC (e.g., Sodium phosphate, polyphosphate or blended phosphate or polyphosphate) is incorporated together with the addition of a buffer (e.g. Hydroxide) in the electrolyte at about 1:250 with respect to FAC content. In various embodiments, other buffering components such as electrochemically generated Hydroxide solutions or DIC buffers, are not employed, or are minimally employed. For example, for solutions having an FAC content of from about 200 ppm to about 250 ppm, sodium phosphate, polyphosphate or blended phosphate or polyphosphate solutions of alkali or alkaline earth material may be incorporated at an amount of from about 5 ppm to 15 ppm to stabilize the solution. In certain embodiments, such solutions are stabilized by incorporating from 1 ppm to about 100 ppm of DIC.

Without being bound by theory, Dissolved Ionic Compounds (DIC), which generally includes sodium, potassium, magnesium, calcium, carbonates, phosphates, bicarbonates, and hydroxides, provides low or minimal buffering capacity in the pH range targeted by the solutions and compositions described herein. Nevertheless, these solutions are effectively stabilized, such that the solutions and compositions are not dependent on “on-demand” production. The stabilizing effect can be due to, in-part, free radical scavenging ability of DIC to thereby slow the decomposition of HOCl. Further still, solutions prepared by electrochemical treatment of hydroxide enriched sodium chloride solution (as opposed to chemical acidification of sodium hypochlorite stabilized with equal amount of hydroxides), have distinct properties with respect to DIC, and the stabilizing effect can be distinct.

While the Hypochlorous Acid solution may be in the form of a liquid, the solution may take the form of a cream, gel (e.g. silicon-based gel), and/or foam by the addition of conventional ingredients known in the art. For example, topical formulations of electrochemical solutions are disclosed in U.S. Provisional application Ser. No. 10/916,278 which is hereby incorporated by reference in its entirety. In these embodiments, the formulation is better contained around the application site by limiting solution run-off. Further, convenient applicators for creams, foams, and the like are known, and may be used in accordance with the present invention. Since the solutions of the invention has a very low conductivity and osmolarity, even with relatively high FAC content, and at “skin-friendly” pH levels, the solutions of the invention are particularly suitable for hydrogel formulations.

In certain embodiments employing hydrogel formulations, the composition has an FAC content of greater than about 100 ppm, greater than about 150 ppm, greater than about 200 ppm, greater than about 250 ppm, or greater than about 300 ppm. Further, the formulation may have a viscosity of from about 0.5 mS/cm to about 12 mS/cm, such as from about 1 mS/cm to about 10 mS/cm in some embodiments. Further, hydrogel formulations in some embodiments have a pH of from about 5 to about 7, or from about 5 to about 6.5 in other embodiments. The stabilized solutions may be packaged for storage or sale, using any suitable container, such as any suitable plastic or glass bottles, or bags (e.g., plastic bags), tubes, or cans (e.g., spray or aerosol). In certain embodiments, the packaging material has minimal gas permeability, including by species such as CO₂ and O₂. The containers may be transparent, or opaque so that they are impenetrable by light, and may be of any unit volume, such as about 100 ml, about 125 ml, about 250 ml, about 0.5 liter, about 1 liter, about 5 liters, about 10 liters, or greater.

The Hypochlorous Acid solution of the invention may also be hypertonic, hypotonic, or isotonic with respect to physiological fluids (blood, plasma, tears, etc.). Alternatively, the solution may contain varying levels of salinity, such as from 0.01 to about 2.0%. Generally, the solution contains from about 0.02% to about 0.9% w/v NaCl when intended for use in medicine. In some embodiments, the solution may be a normal saline solution (about 0.9% w/v NaCl). In some embodiments, the solution may contain from about 0.01 to 2.0% w/v one or more salts, such as e.g. NaCl, KCl, or a mixture of salts. The salt may be a salt of an alkali metal or alkaline earth metal, such as sodium, potassium, calcium, or magnesium.

In another aspect, the invention provides a method for preparing the stabilized Hypochlorous Acid solution. The method involves incorporating an electrochemically generated hydroxide into an electrolyte for electrochemical treatment, or directly to an electrolyzed solution comprising Hypochlorous Acid.

For example, an electrochemically generated Hypochlorous Acid solution may be diluted with water or aqueous solution comprising electrochemically generated hydroxides or DIC. In other embodiments, the electrochemically generated Hypochlorous Acid solution (e.g., having the desired FAC content) is added to containers comprising sodium phosphate, polyphosphate or blended phosphate or polyphosphate of alkali or alkaline earth material. The latter is an effective method for production of low ionic strength Hypochlorous Acid solutions, especially for hydrogel formulations.

The stabilized Hypochlorous Acid solutions (e.g. solutions of greater than 90%, 95%, or 97% HOCl) may be obtained by electrolysis of a brine solution as described in U.S. Pat. Nos. 7,276,255; 7,691,249; 7,374,645, which is hereby incorporated by reference in its entirety, or can be prepared by any suitable method or apparatus, by incorporating the DIC into the electrolyte or the solution for electrolysis. Hypochlorous Acid solutions may be prepared by passing brine solution containing electrochemically generated hydroxide and/or DIC through one or more electrolytic cells as described, for example, in U.S. Pat. No. 7,303,660, U.S. Pat. No. 7,828,942, and U.S. Pat. No. 7,897,023, which are hereby incorporated by reference.

Still other aspects of the invention provide methods of disinfecting or cleansing a mammalian tissue, such as a wound or burn, or disinfecting or cleansing a hard surface, or for treating or preserving a food product or cut flowers. Due to the stability of the Hypochlorous Acid solutions, such methods need not be performed proximately to the production of the biocidal solution, and the solution may be prepared well in advance of its use.

The solutions and formulations of the invention may be used as a sterilizing, disinfecting and biocidal solution for human and animal care. The solutions are non-hazardous, non-irritating, non-sensitizing to the skin, non-irritating to the eyes, not harmful if swallowed, and show no evidence of mutagenic activity. For example, the method of the invention provides for moistening, lubricating, irrigating, cleaning, deodorizing, disinfecting, or debriding a wound by rinsing, washing or immersing the wound, with or in, the stabilized or stored Hypochlorous Acid solutions, or by applying the solution to the wound and/or wound dressing. The wound may or may not be infected, and thus the method of the invention is useful for treating infected wounds and useful for preventing infection of uninfected wounds.

In one aspect, the invention provides a convenient means for wound care treatment applying the stabilized solution to a wound site by one or more of soak, scrub, pulsed lavage, hydro surgery, and ultrasound to effectively debride and disinfect a wound or tissue. The solution may be delivered before, during and/or after negative pressure wound therapy to promote proper wound healing physiology. In these embodiments, the method may employ a wound dressing for coordinating debridement by infusion of Hypochlorous Acid with negative pressure therapy. Thus, the invention may be used in combination with a wound treatment apparatus and/or wound dressing.

For example, in certain embodiments, the invention allows for an initial stabilized Hypochlorous Acid solution soak and/or scrub to both debride and disinfect the wound or tissue, followed by the application of negative pressure to the wound or tissue (as described herein) using the stabilized Hypochlorous Acid solution as an irrigant to control wound bio burden, remove excess exudate, and promote formation of granulation tissue. Optionally, the method also involves seamless transition to the stabilized Hypochlorous Acid solution infusion (e.g., active or passive infusion without negative pressure). Such seamless transition can be effected via a wound dressing which allows for controlled infusion of stabilized Hypochlorous Acid solution with controlled vacuum source. In this embodiment, continued cell proliferation and regeneration continues without disruption of the wound bed, once the endpoints of negative pressure therapy have been obtained.

In certain embodiments of the invention, the wound needing care is a stage I-IV pressure ulcer, stasis ulcer, diabetic ulcer, post-surgical wound, burn, cut, abrasion, or a minor irritation of the skin. In certain embodiments, the wound is rinsed, washed, or immersed in the solution periodically over at least two weeks, but treatment may continue periodically for over about 4 weeks, about 9 weeks, or more. The wound, in some embodiments, is rinsed with the solution at least once a week, but may be treated with the solution at least twice a week, or more frequently.

While the Hypochlorous Acid solution may be delivered to the wound at room temperature, the solution may alternatively be heated, for example, to body temperature or about body temperature. In this embodiment, the solution is comfortable and soothing for the patient, and is more effective.

In some embodiments, the invention provides a method for treating an infected or colonized wound, tissue, surgical cavity, or bone, and a method for reducing wound bio burden. The treatment solution in accordance with the invention, as already described, is generally effective for killing or inactivating a broad spectrum of bacterial, fungal, and viral pathogens, including S. aureus, P. aeruginosa, E. coli, Enterococcus spp., C. difficile, and Candida Spp. The solution does not produce resistant species, making the methods desirable over the delivery of traditional antibiotics.

In another aspect, the solution of the invention is particularly suitable for use in conjunction with stern cell and growth factor therapy, including the use of genetically engineered cells and engineered tissue and allografts and organs for transplant in various treatments. Using the stabilized Hypochlorous acid solution of the invention to disinfect tissue before, during or after addition of cells or growth factors, maintains the viability of the cells and integrity of the growth factors, while killing the unwanted microbes.

In certain embodiments, the solution or formulation thereof is applied for the control of inflammation, including an inflammatory reaction or hyper inflammation of the skin. For example, the solution or formulation thereof may be applied for use in a method as described in U.S. patent application Ser. No. 11/656,087 or 12/523,507, which are hereby incorporated by reference. In certain embodiments, the solution or composition of the invention is applied (e.g., to an effected area) for treatment of a patient having a dermatoses, atopic dermatitis, skin allergy, rosasea, psoriasis, or acne, among others. In such embodiments, the HOCl solution may be formulated as a hydrogel, for example, as described elsewhere herein.

In certain embodiments, invention is advantageous for use against microbes on surfaces because of its fast activity against bacterial spores, fungi, and other resistant microorganisms. Because of its effectiveness and the speed at which it acts, the invention meets a substantial public health need, and one that is not adequately addressed by current commonly-used antimicrobial agents. Accordingly, application of the solution to various surfaces and materials is useful to control microbial contamination, not only for the care and management of wounds, but for disinfecting hard surfaces such as medical or dental equipment, preserving and decontaminating food products, water treatment, as well as other industrial and agricultural applications. In certain embodiments, the solution or composition of the invention is applied to crops (pre- or post-harvest) or cut flowers for their preservation and/or for improving the overall quality of the product. In some embodiments, the solution is potassium based and has one or more utilities (e.g., methods of use) disclosed in U.S. patent application Ser. No. 13/423,822, which is hereby incorporated by reference in its entirety.

In various embodiments, including the treatment of food, agricultural products, and surfaces the solution can be applied as a mist, fog, spray, or ice. Killing, inactivating, or otherwise reducing the active population of bacterial spores and fungi on surfaces is particularly difficult. Bacterial spores have a unique chemical composition of spore layers that make them more resistant than vegetative bacteria to the antimicrobial effects of chemical and physical agents. Likewise, the unique chemical composition of fungal cells, especially mold spores, makes them more resistant to chemical and physical agents than are other microorganisms. This resistance can be particularly troublesome when the spores or fungi are located on surfaces such as food, food contact sites, ware, hospitals and veterinary facilities, surgical implements, and hospital and surgical linens and garments.

Control of the mold Chaetomium limicola, and of bacterial spore-forming microorganisms of the Bacillus species, can be especially important during food packaging, particularly during cold or hot aseptic filling of food and beverage products. Microorganisms of the Bacillus species include Bacillus cereus, Bacillus mycoides, Bacillus subtilis, Bacillus anthracis, and Bacillus thuringiensis. These latter microorganisms share many phenotypical properties, have a high level of chromosomal sequence similarity, and are known enterotoxin producers. Bacillus cereus is one of the most problematic because Bacillus cereus has been identified as possessing increased resistance to germicidal chemicals used to decontaminate environmental surfaces.

As used herein, the term “surface” refers to both hard and soft surfaces and includes, but are not limited to, tile grout, plaster, drywall, ceramic, cement, clay, bricks, stucco, plastic, wallpaper, fabric, tiles, cement, and vinyl flooring, heating and/or cooling fins, filters, vanes, baffles, vents, crevices in walls or ceilings, paper and wood products such as lumber, paper, and cardboard, woven products such as blankets, clothing, carpets, drapery and the like. The term surface also includes human surfaces, animal surfaces, military equipment, transportation equipment, children's items, plant surfaces, seeds, outdoor surfaces, soft surfaces, air, wounds, and medical instruments, and the like.

In chemistry, the Bronsted-Lowry theory is an acid-base reaction theory. Theory states an acid is defined the ability to “donate” a proton (H⁺) and a base is defined as a species with the ability to gain, or “accept,” a proton. A sodium chloride brine solution is an extremely very weak Bronsted-Lowry base (Cl) and Bronsted-Lowry acid (Na⁺). It is a neutral solution resulting in minimal movement of protons between ions. The lack of proton exchange across the membrane in the electrochemical cell causes a constant deviation in pH and free available chlorine concentration. Increasing the Bronsted-Lowry base (Cl−) by acidifying the brine creates a downward pH shift (2.8 to 4.0) resulting in higher percentages of chlorine gas and less hypochlorous acid. Increasing the Bronsted-Lowry acid (Na+) by adding an alkalizing the brine create an upward pH shift. This is the preferred pH range for the product (5.5 to 7.0). Within this pH range, we maximize Hypochlorous acid concentration and minimize corrosion potential of the solution.

Le Chatelier's principle or “the common ion effect” can be used to predict the effect of a change in conditions on an equilibrium solution. The common ion effect is defined as the suppression in the degree of dissociation of a weak electrolyte containing a common ion. The attraction between the Na⁺ and Cl⁻ ions in the solid is so strong that only highly polar solvents like water dissolve NaCl well. When dissolved in water, the sodium chloride framework disintegrates as the Na⁺ and Cl⁻ ions become surrounded by the polar water molecules. These solutions consist of metal aquo complex with the formula [Na(H₂O)₈]⁺, with the Na—O distance of 250 picometers. The chloride ions are also strongly solvated, each being surrounded by an average of 6 molecules of water. These polar water molecules insulate the Na+ and Cl− ions preventing proton movement. By changing the degree of dissociation of the sodium chloride brine solutions, the number of polar molecules insulating the Na+ and Cl− ion is reduced enabling proton movement between ions.

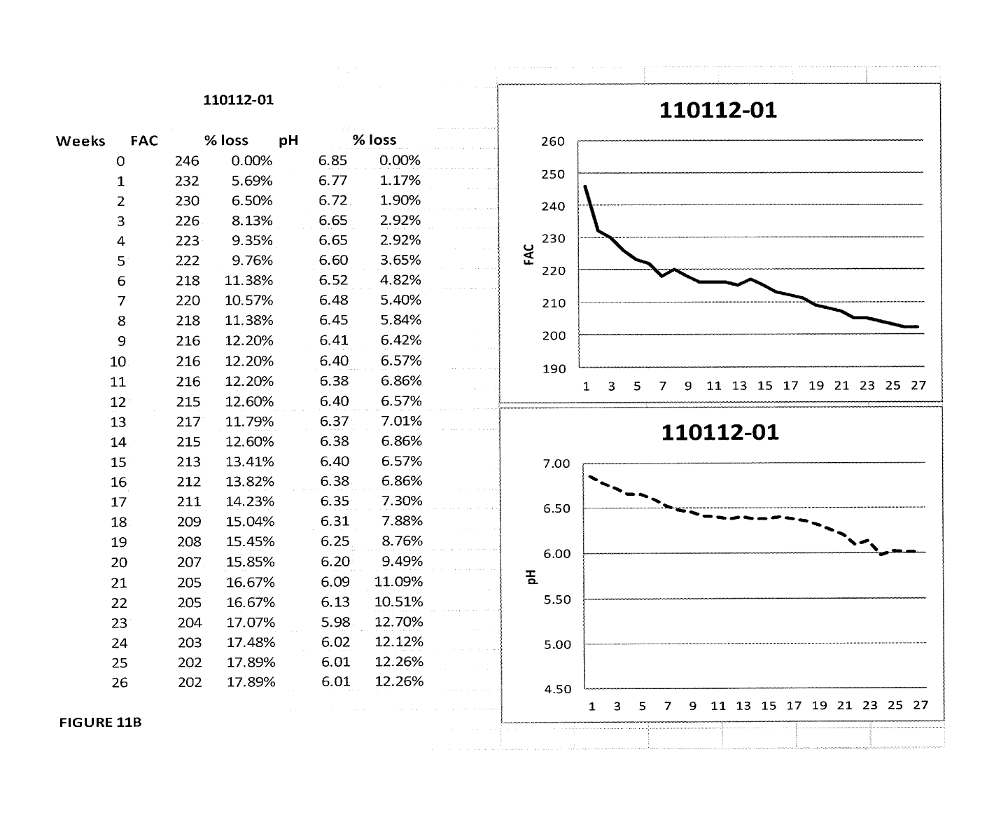

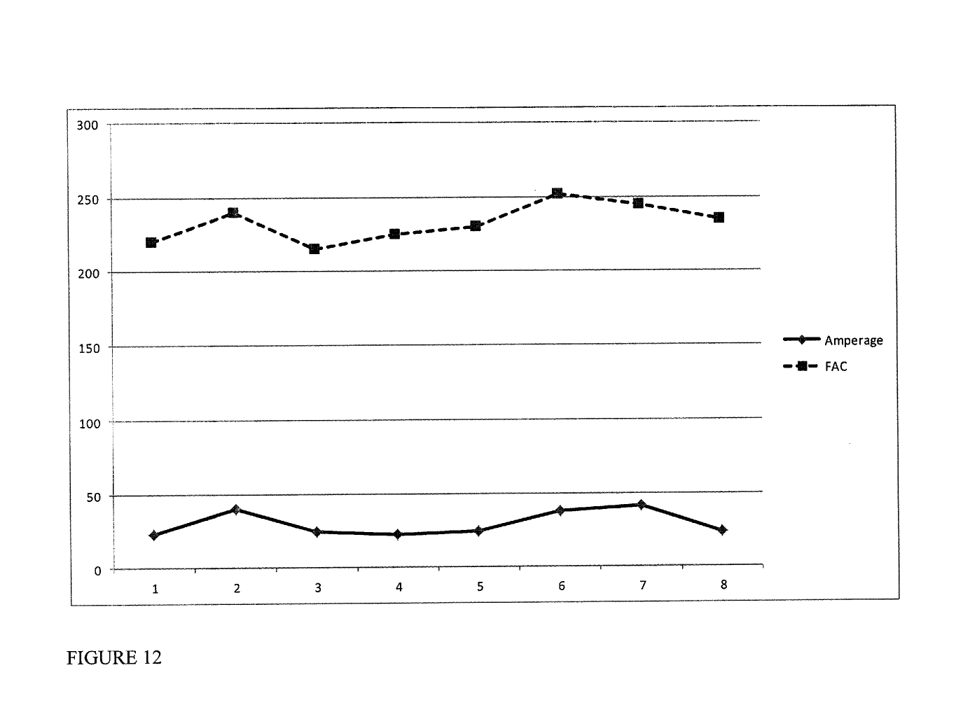

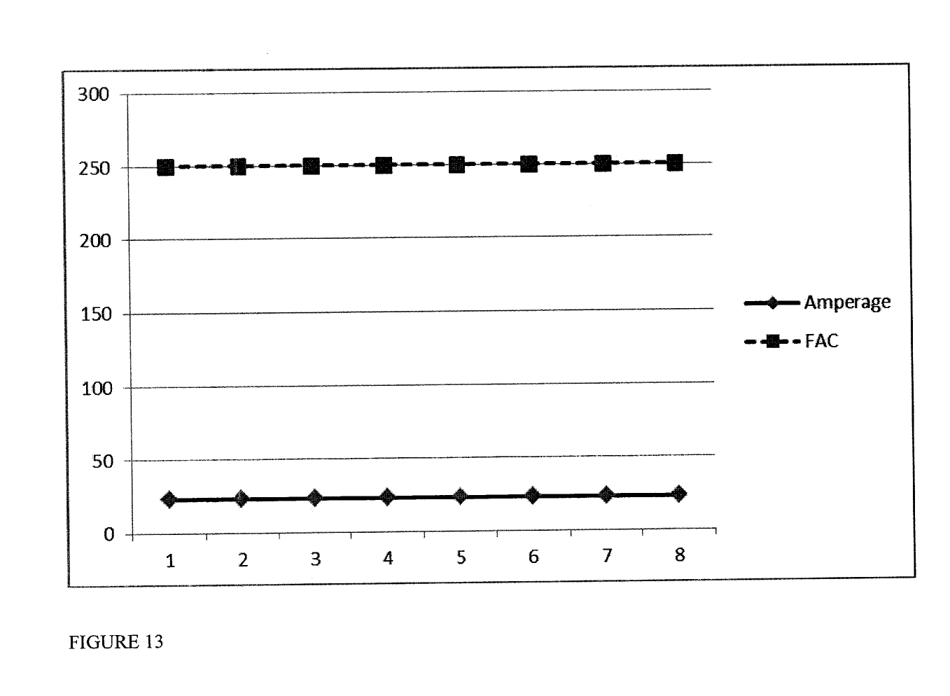

The present invention uses sodium hydroxide's “common ion effect” of the Na+ ion to change the dissociation ratio of the sodium chloride. The increase in Na+ ion concentration drives the electrolysis reaction inside the cell toward the cathode. This means the proton (H+) movement is driven toward the anode creating consist amperage on the anode side of the electrochemical cell. The degree of dissociation required to change the reaction dynamics is minimal. Increasing the sodium chloride brine solution's pH to 9.0, the metal aquo complex loses more than 50% of the polar water molecules enable the Na+ ions to donate protons. FIG. 12 is a chart to depict the effect of electrochemically generated Hydroxide addition in the electrolyte on pH and solution stability in the electrochemically generated HOCL solution—Amperage vs. FAC (Sodium Chloride Brine pH=7.0). FIG. 13 is a further chart to depict the effect of electrochemically generated Hydroxide addition in the electrolyte on pH and solution stability in the electrochemically generated HOCL solution—Amperage vs. FAC (Sodium Chloride Brine pH=9.0).

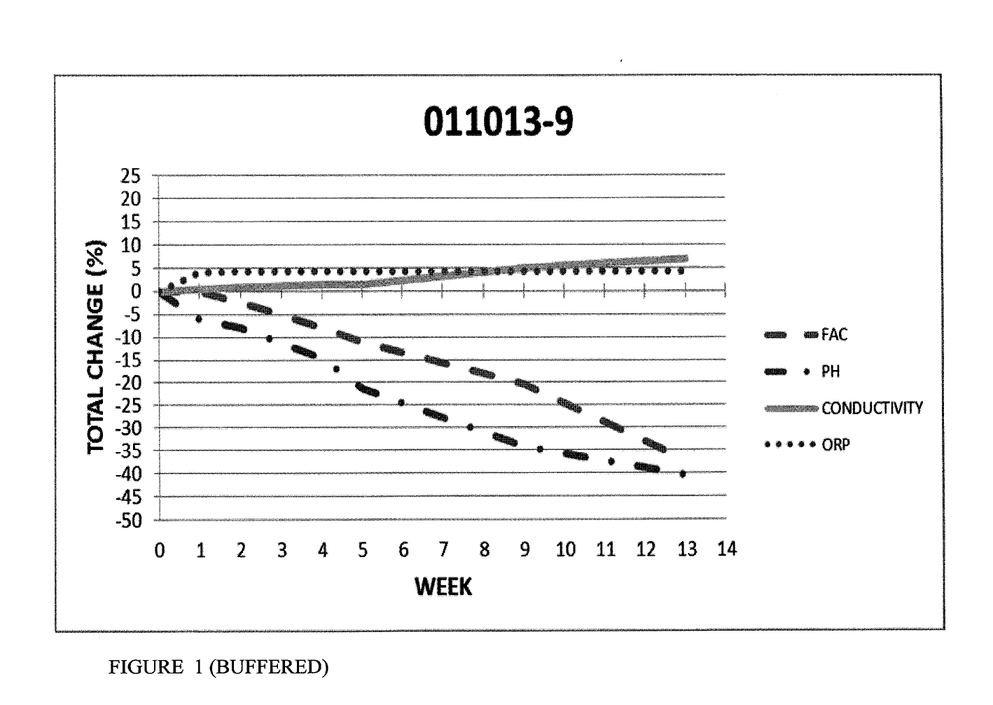

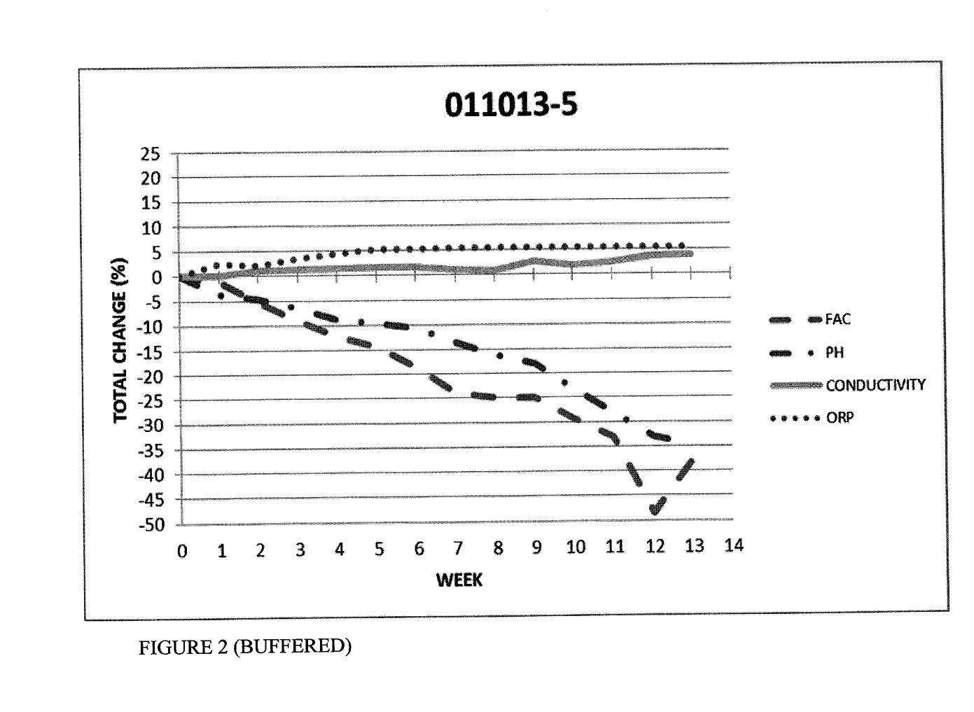

FIGS. 1 and 2 are charts that depict the change in pH and FAC over time of a stabilized electrochemically HOCl solution (buffered) stored in unsealed (spray cap) HDPE bottles at 40 degrees Celsius and 75% relative humidity.

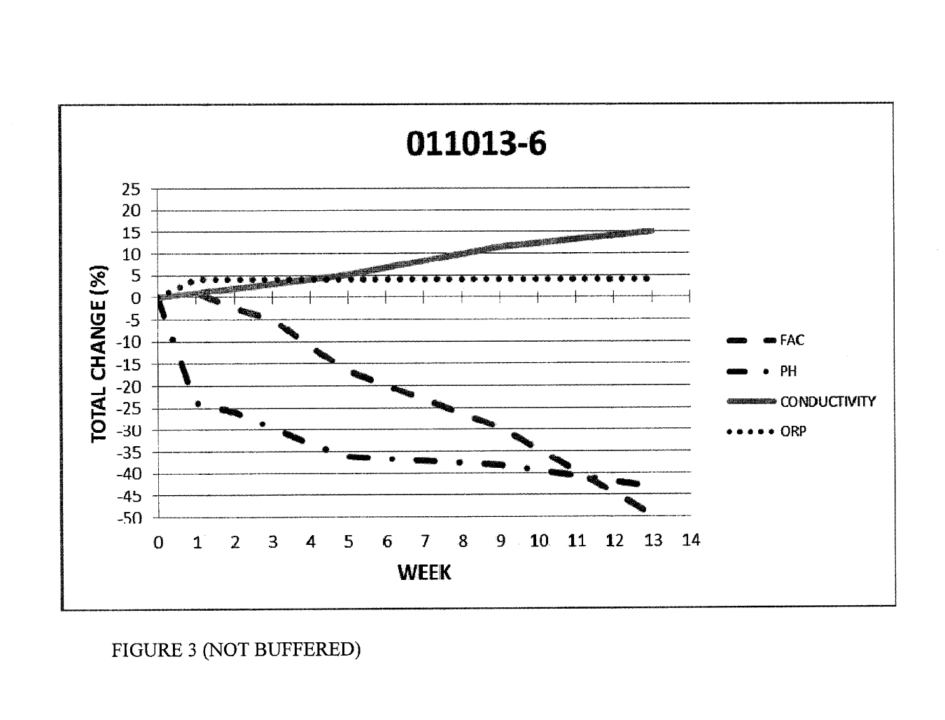

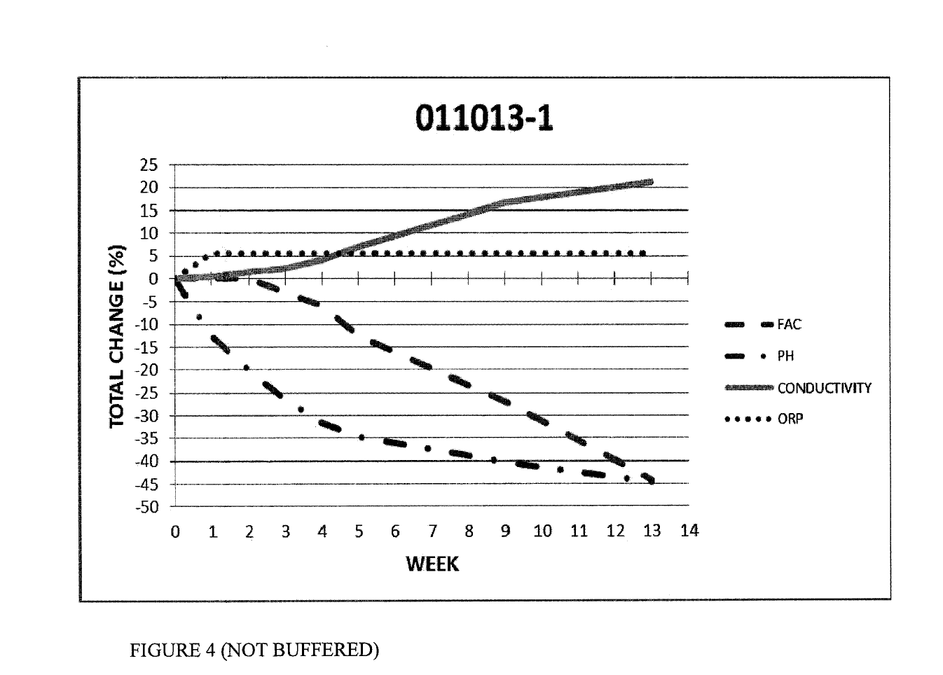

FIGS. 3 and 4 are charts that depict the change in pH and FAC over time of an electrochemically generated HOCl solution (non-buffered) stored in unsealed (spray cap) HDPE bottles at 40 degrees Celsius and 75% relative humidity.

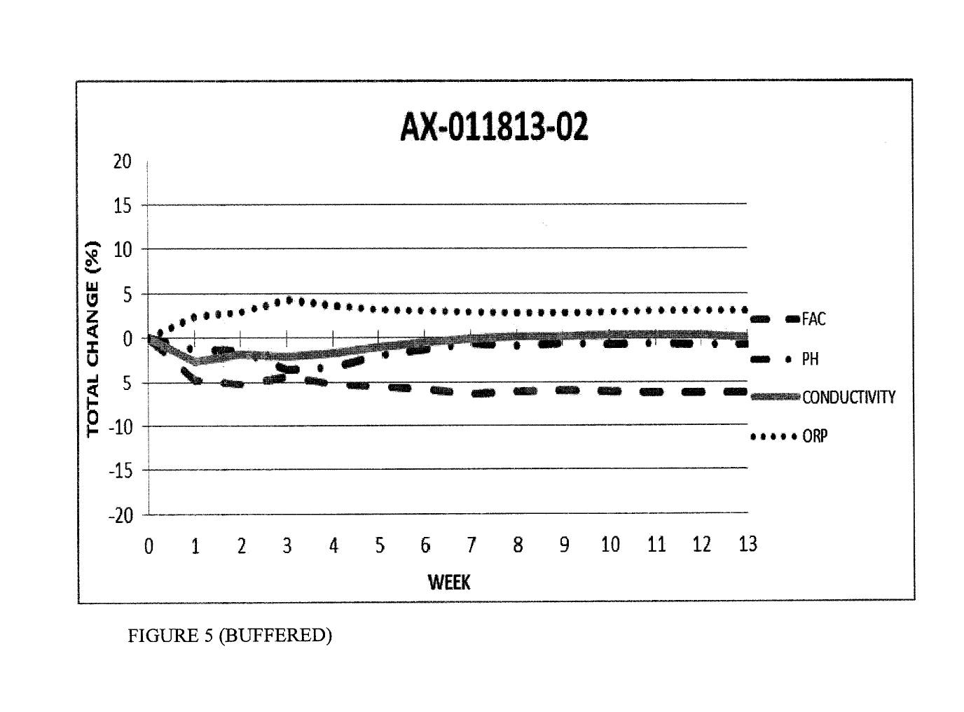

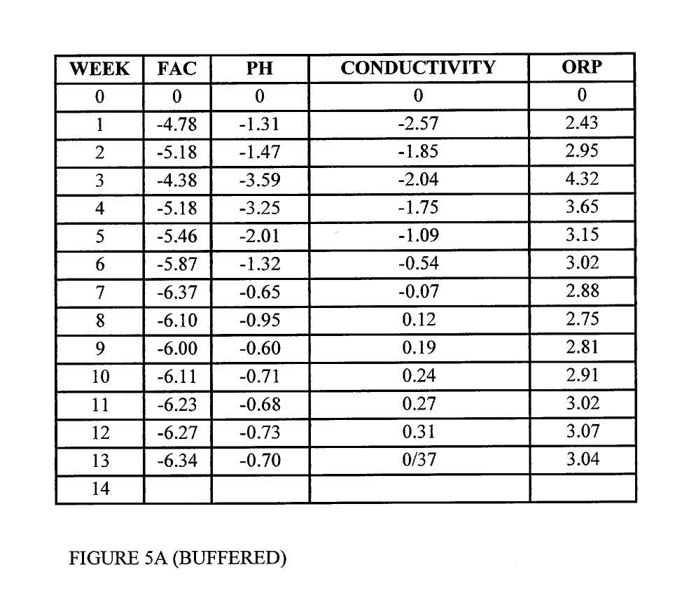

FIG. 5 is a chart that shows the change in pH and FAC over time of a stabilized electrochemically generated HOCl solution (buffered) stored in sealed Jerri cans, drums and totes at ambient temperature.

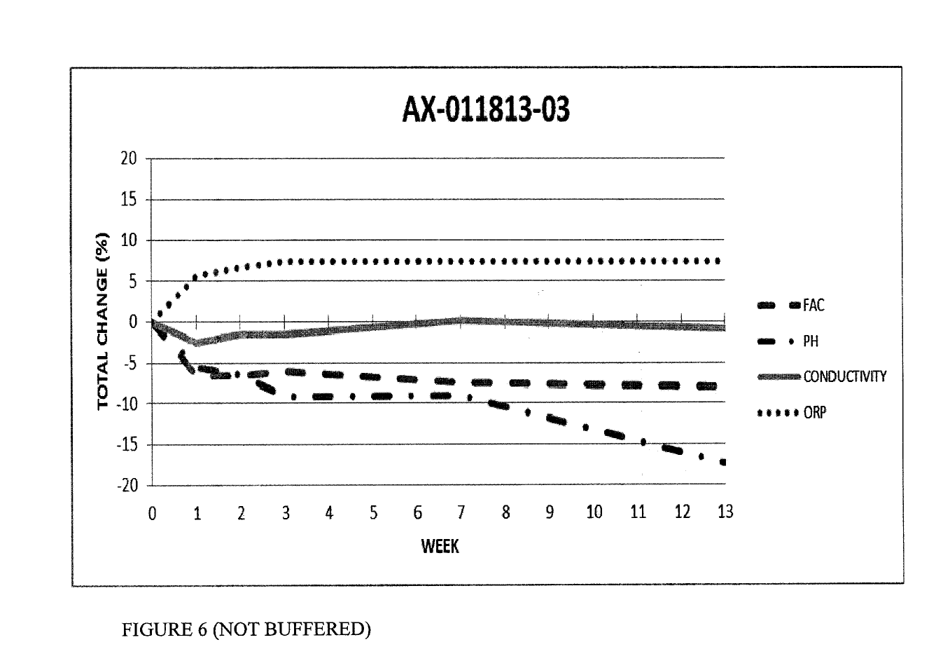

FIG. 6 is a chart that depicts the change in in pH and FAC over time of an electrochemically generated HOCl solution (non-buffered) stored sealed Jerri cans, drums and totes at ambient temperature

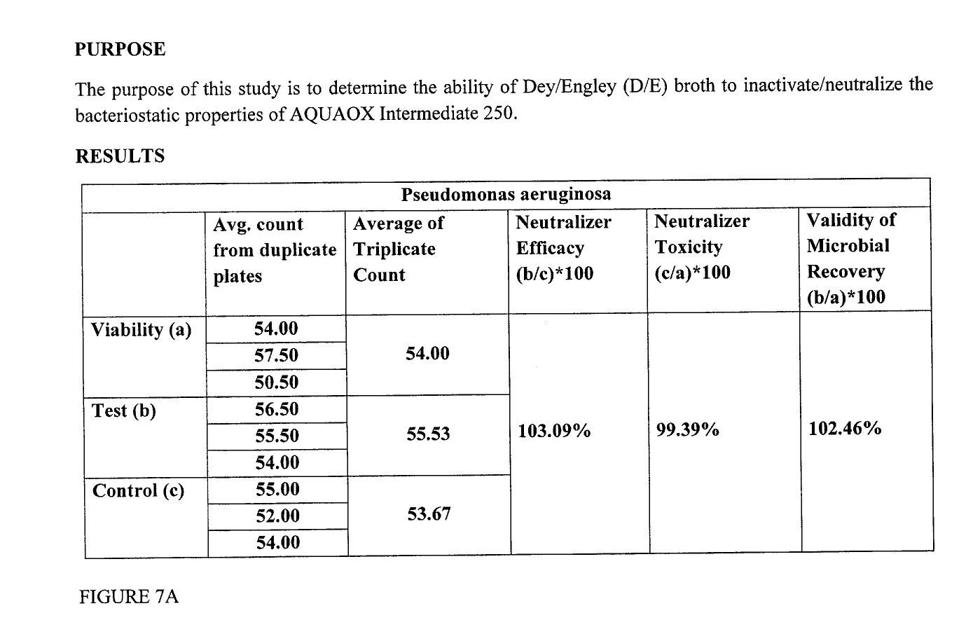

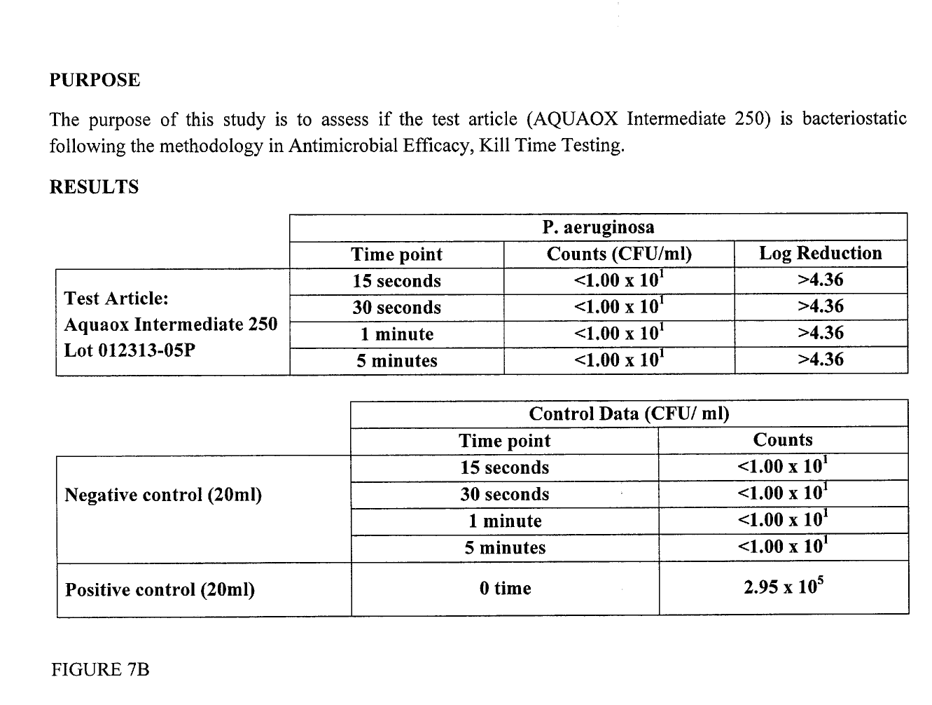

FIG. 7 depicts the efficacy of a stabilized electrochemically HOCl solution (buffered).

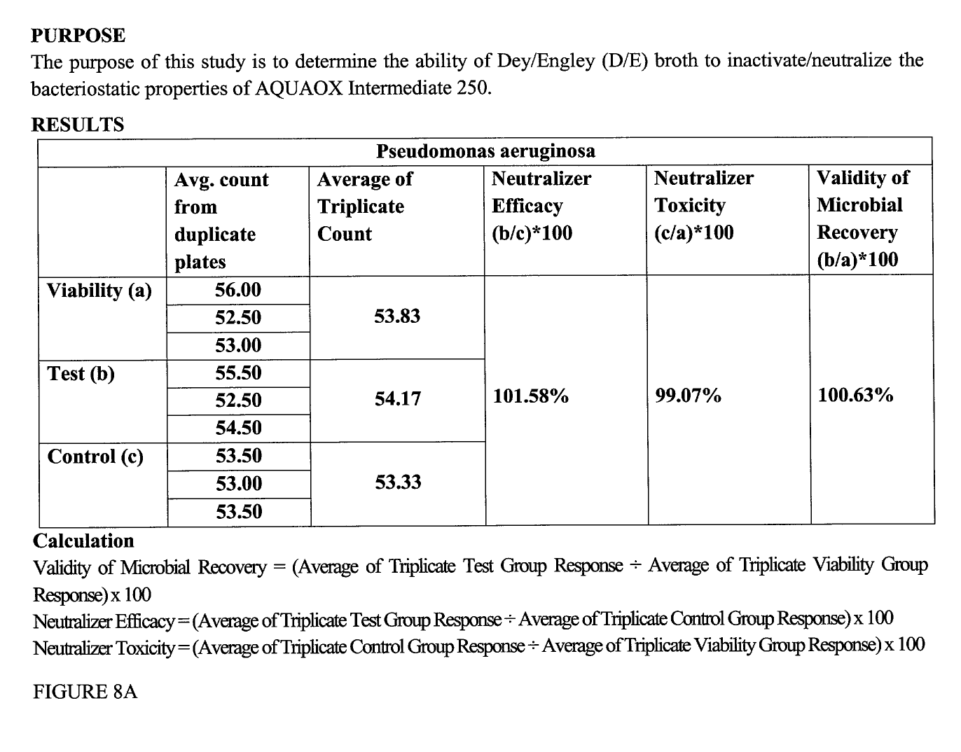

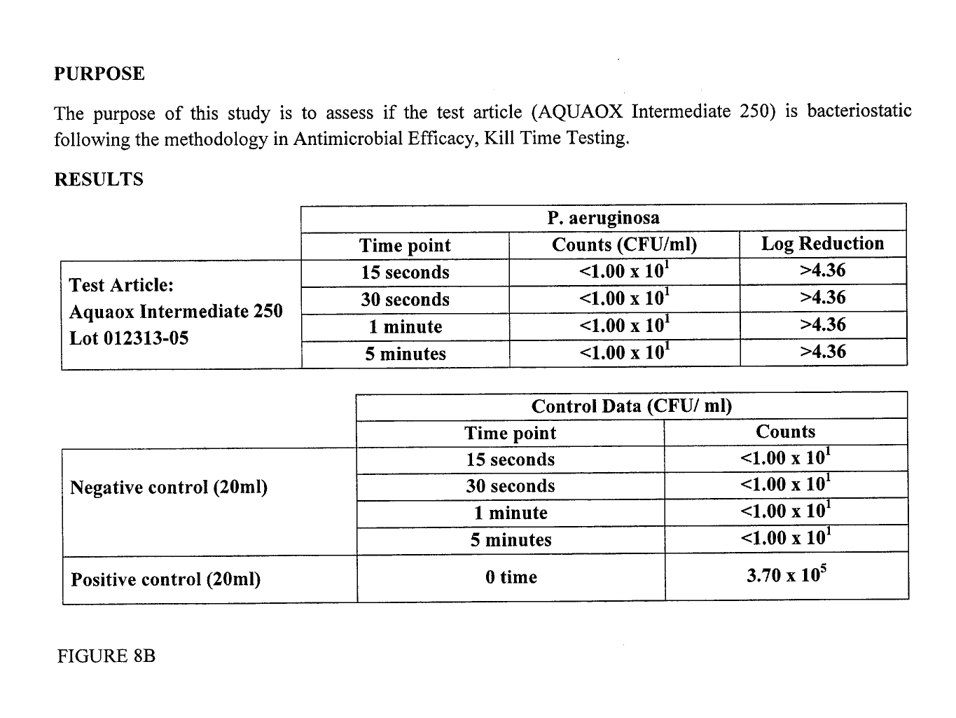

FIG. 8 depicts the efficacy of an electrochemically generated HOCL solution (non-buffered).

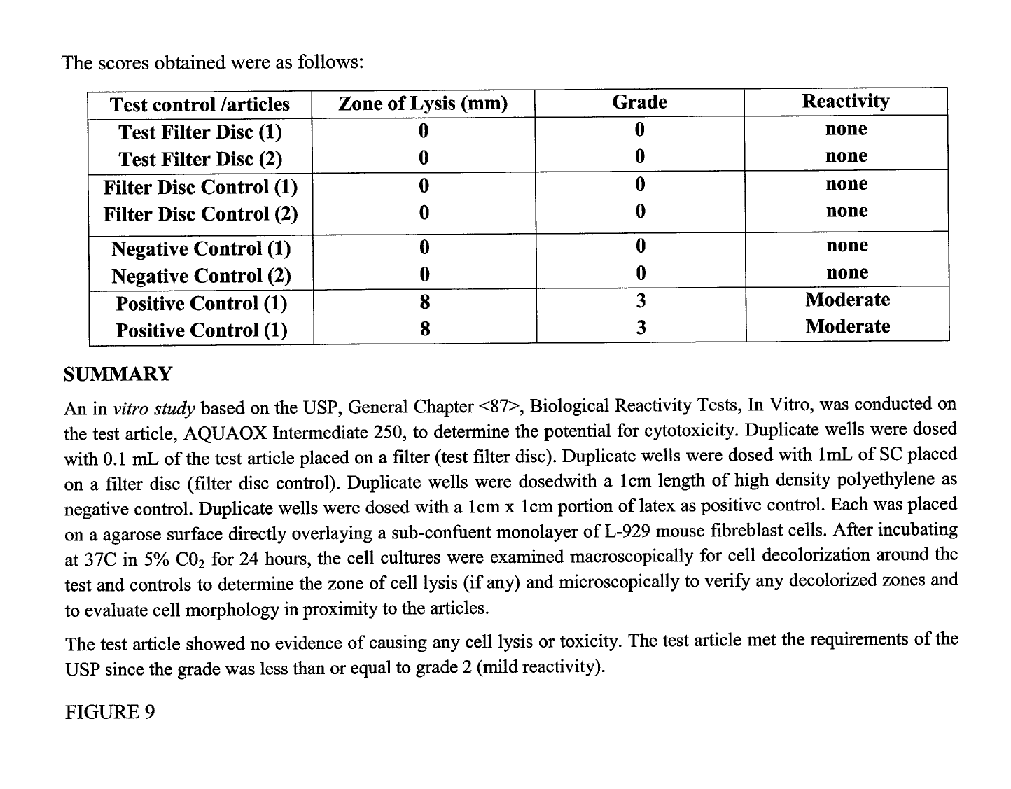

FIG. 9 shows the toxicity of a stabilized electrochemically HOCl solution (buffered).

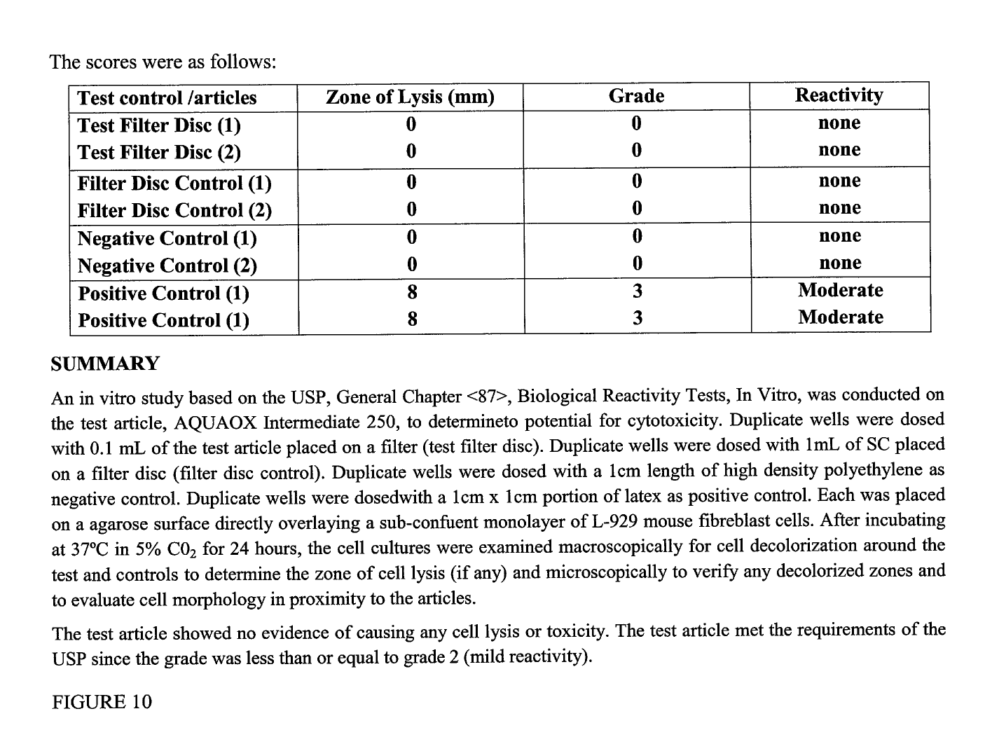

FIG. 10 shows the toxicity of an electrochemically generated HOCl solution (non-buffered).

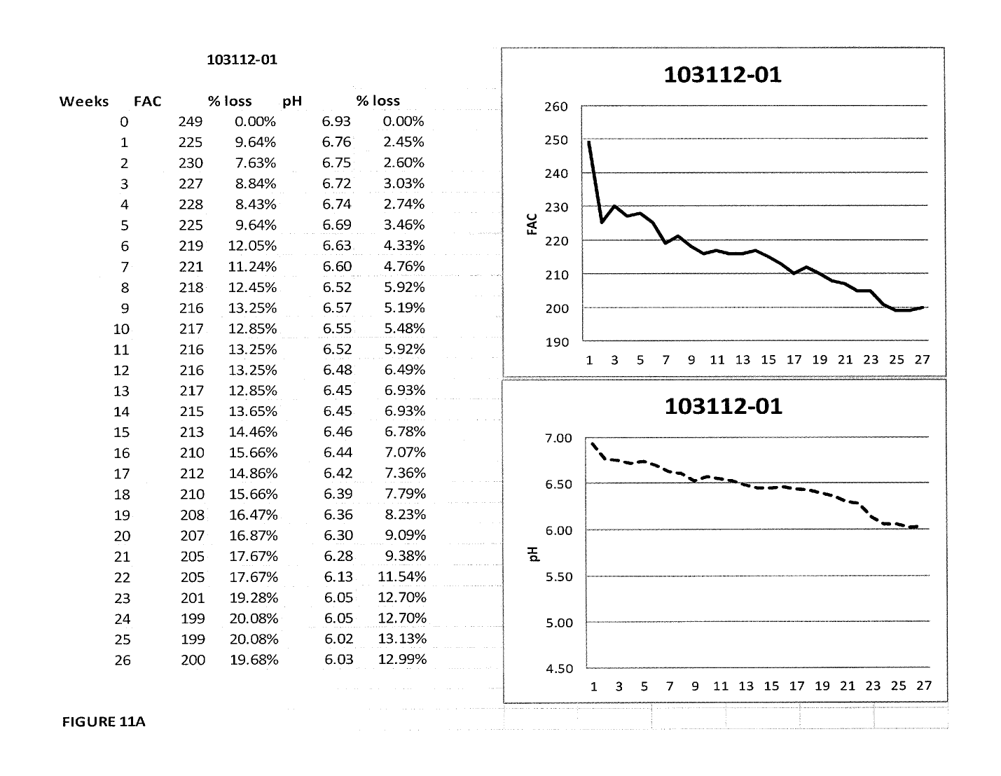

FIG. 11 is a chart and graphs of the extended stability of samples of batches stabilized (buffered) electrochemically generated Hypochlorous Acid solutions.

FIGS. 12 and 13 depict the effect of electrochemically generated Hydroxide addition in the electrolyte on pH and solution stability in the electrochemically generated HOCL solution. It is very common in the chlor-alkali industry to acidify the brine solution to improve the cell current efficiency. The present invention uses a moderately alkaline brine to enhance cell current efficiency and minimize the variation in pH and FAC in the final anolyte solution.

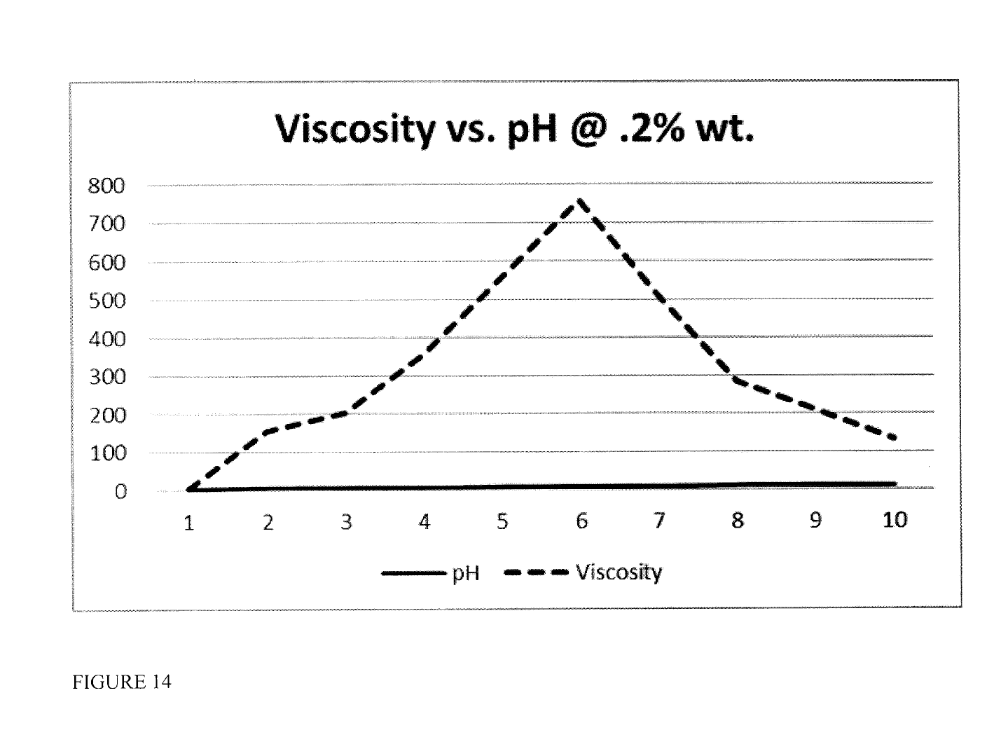

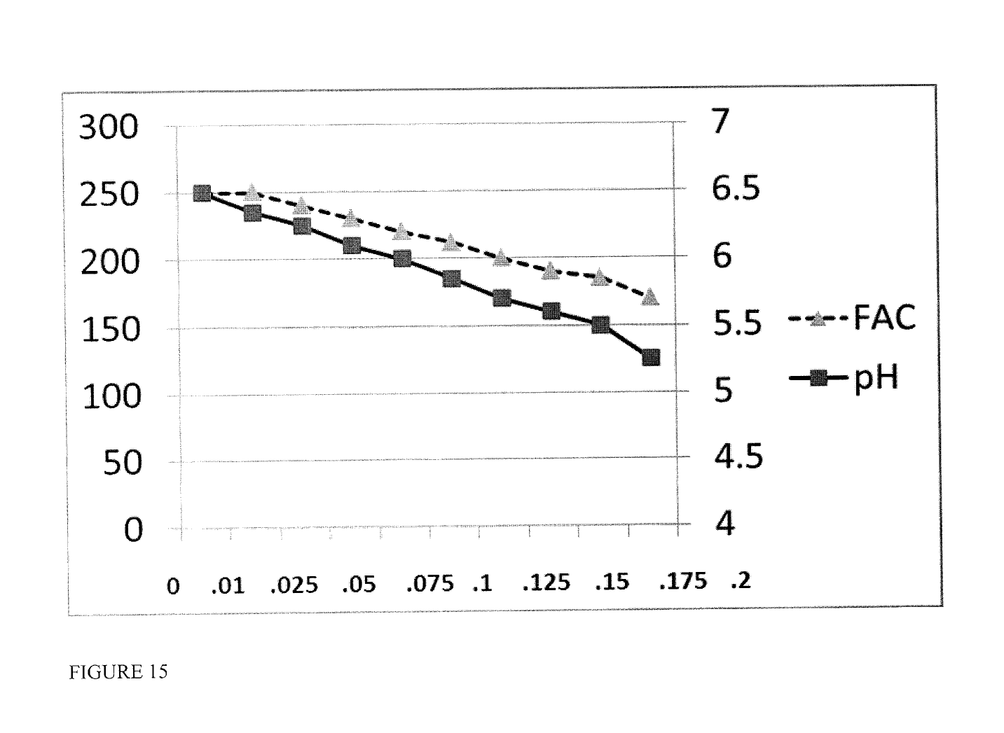

FIG. 14 shows a shift in pH upon formulation as a hydrogel. The present invention uses a highly crosslinked polyacrylate powder to develop thickening, stabilization and suspension properties to the hypochlorous acid solution. The thickened hypochlorous acid product called Hydrogel, use 0.2% by weight of the polyacrylate powder. The Hydrogel product specification for viscosity is in the range of 100 to 550 centipoise. The polyacrylate powder's thickening properties are dependent on the solutions pH. Below is a graph showing the relationship between solution pH and viscosity. Since solution pH is critical to developing viscosity, we must calculate the FAC (free available chlorine) drop and pH shift during polyacrylate addition. FIG. 15 illustrates the resultant FAC (free available chlorine) and pH as various concentrations of polyacrylate are added:

### Example 1

### Accelerated Stability Study: Comparison of Stability of HOCL Solutions with or without DIC

FIGS. 1 and 2 are graphs with FAC, pH, ORP and Conductivity measurements for Hypochlorous Acid wound treatment solutions as a function of time under accelerated stability conditions (40 degrees Celsius at 75% relative humidity).

FIGS. 3 and 4 are graphs with FAC, pH, ORP and Conductivity measurements for Hypochlorous Acid wound treatment solutions as a function of time under accelerated stability conditions (40 degrees Celsius at 75% relative humidity).

In an attempt to stabilize the pH and Conductivity, the samples in FIGS. 1 and 2 were buffered with a blend of sodium phosphate and polyphosphates. The results show that the pH of the samples containing a buffer were more stable (<35% loss in 12 weeks) when compared to non-buffered (>40% loss in 12 weeks) samples. FAC of the buffered samples were more stable (<35% loss) than the FAC of non-buffered samples (>40% loss).

The stability of the stabilized solution as a function of time was tested. Hypochlorous Acid was produced by electrochemical treatment of a brine solution. The solution had a pH of 5.5 to 6.5, a conductivity of approximately 1250 uS, and Osmolarity of less than 50 Osm/L. and 200 to 250 ppm of FAC. This solution was packaged in HDPE bottles and stored 40 degrees Celsius at 75% relative humidity. The biocidal activity and stability of the solution as a function of time was tested by measuring FAC, pH, ORP and Conductivity (Osmolarity) content in unopened unsealed (spray cap) HDPE test bottles over a period of 12 weeks.

12 weeks of accelerated stability is comparable with 66 weeks stability under normal (ambient) storage conditions. The accelerated stability is based on a “first order rate law”. This rate law predicts concentration change of hypochlorous acid over time. The rate law for a reaction that is first order with respect to our reactant hypochlorous acid is:

$$r = - \frac{d [ A ]}{d t} = k [ A ]$$

Hypochlorous Acid degradation rate (r)) equals the change in hypochlorous acid concentration from initial production to the end of two years (d[A]). Temperature changes can be measured but all stability samples are kept at constant temperature. The above equation can be expressed as:

ln [A]=−kt+ln [A]₀

Subtracting the natural log of our initial concentration by the natural log of our 2 year concentration, we find the change in hypochlorous acid concentration. Since the time (t) is known (2 years), we solve for (k) hypochlorous acid degradation rate. This is our rate constant of hypochlorous acid at this specific concentration. Note: Hypochlorous acid will have a different degradation rate at varying concentrations. However, this same “first order rate law” applies to all concentrations. Knowing the rate constant of hypochlorous acid at any given concentration, we can calculate concentrations at any time or temperature. Using the rate constant of a 250 ppm hypochlorous acid solution and increasing the temperature to 40 degrees Celsius, we have found that 7 days equals 41 days at ambient temperature. The results, showing that the solutions are stabilized, with regard to FAC content and pH for over one year.

### Example 2

### Ambient Stability Study: Comparison of Stability of HOCL Solutions with or without DIC

The stability of the stabilized solution as a function of time was tested. Hypochlorous Acid was produced by electrochemical treatment of a brine solution. The solution had a pH of 5.5 to 6.5, a conductivity of approximately 1250 uS, and Osmolarity of less than 50 Osm/L. and 200 to 250 ppm of FAC. This solution was packaged in HDPE 5 gallon jerry cans, 100 liter drums and 1000 liter bulk-containers and stored at ambient temperature. The biocidal activity and stability of the solution as a function of time was tested by measuring FAC, pH, ORP, Conductivity and Osmolarity content in unsealed test bottles over a period of 26 weeks.

FIG. 5 is a graph with FAC, pH, ORP, Conductivity and Osmolarity measurements for Hypochlorous Acid wound treatment solutions as a function of time under normal storage conditions (ambient temperature).

FIG. 6 is a graph with FAC, pH, ORP, Conductivity and Osmolarity measurements for Hypochlorous Acid wound treatment solutions as a function of time under normal storage conditions (Ambient temperature).

In an attempt to stabilize the pH and Conductivity, the samples in FIG. 5 were buffered with a blend of sodium phosphate and polyphosphates.

The results show that the pH of the samples containing a buffer were considerably more stable (˜1% loss) when compared to non-buffered (˜17% loss in 12 weeks) samples. The FAC of the buffered samples were slightly more stable (˜6% loss) than the FAC of non-buffered samples (˜8% loss in 12 weeks). The results, showing that the solutions are stabilized, with regard to FAC content and pH for at least 3 months.

### Example 3

### Microbial Efficacy Study: Comparison of Antimicrobial Properties of HOCL Solutions with or without DIC

Three different batches comprising a buffered electrochemical Hypochlorous Acid solution with a targeted FAC of 250 ppm and 3 different batches comprising a non-buffered electrochemical Hypochlorous Acid solution with a targeted FAC of 250 ppm were produced. Samples of the buffered and non-buffered batches were analyzed to determine and compare the efficacy of buffered and non-buffered Hypochlorous Acid solutions.

Two efficacy tests were conducted for each sample.
a) Validation of Microbial Recovery: the purpose of this study was to determine the ability to inactivate the bacteriostatic properties of the HOCL solution
b) Kill Time evaluation: the purpose of this study is to assess if the HOCL solution is bacteriostatic against Pseudomonas aeruginosa at least through 5 minutes.

FIG. 7 shows the efficacy of stabilized electrochemically HOCL solution (buffered) and FIG. 6 shows the efficacy of electrochemically generated HOCL solution (non-buffered).

Both the buffered and non-buffered samples demonstrated over 99% recover and exceeded the test acceptance criteria (70% recovery). Although not significant all tests showed that the buffered HOCL solutions demonstrated a slightly better microbial recovery than the non-buffered solutions.

Both the buffered and non-buffered samples demonstrated a 4.36 Log reduction against Pseudomons aeruginosa organisms at any time point. The results (FIG. 5 and FIG. 6) demonstrate that the stabilized (buffered) HOCL solution possesses an equal level of biocidal activity against Pseudomonas aeruginosa organisms compared to electrochemically generated (non-buffered) HOCL solutions.

### Example 4

### Cytotoxicity Study: Comparison of Toxicity of HOCL solutions with or without DIC.

The purpose of this study was to determine the potential to cause cytotoxicity and to determine the effect of DIC on cytotoxicity. Hypochlorous Acid solutions were electrochemically generated, and some Hypochlorous Acid solutions additionally buffered with DIC. Compositions of Hypochlorous Acid with and without phosphate additives were tested. The results showed that addition of DIC compared to the FAC amount in the electrochemically generated Hypochlorous Acid solution of about 1:250 has no effect on the cytotoxicity.

The study was conducted based on US Pharmacopeia, National Formulary, General Chapter <87>, Biological Reactivity Tests, In Vitro.
The test filter disc was placed on the solidified agarose surface in two separate cell culture wells. Similarly, the filter disc control, the negative control, and the positive control were each placed on the solidified agarose surface in two cell culture wells. Each cell culture well was incubated at 37° C. in 5% CO₂ for 24 hours.
Following incubation, the cultures were examined macroscopically for cell decolorization around the test article and controls to determine the zone of cell lysis (if any). After macroscopic examination, the cell monolayers were examined microscopically (100×) to verify any decolorized zones and to evaluate cell morphology in proximity to the article.

Scoring for cytotoxicity was based on the following criteria:

| Grade | Reactivity | Condition of Cultures |
|---|---|---|
| 0 | None | No detectable zone around or under specimen |
| 1 | Slight | Some malformed or degenerated cells under |
| | | specimen |
| 2 | Mild | Zone limited to area under specimen and up to |
| | | 4 mm |
| 3 | Moderate | Zone extends 5-10 mm beyond specimen |
| 4 | Severe | Zone extends greater than 10 mm beyond specimen |

For the suitability of the system to be confirmed, the negative control and the filter disc control must have been a grade of 0 (reactivity none) and the positive control must have produced a zone of lysis (reactivity moderate to severe). The test article met the limits of the test if both monolayers exposed to the test article showed no greater than a grade of 2 (reactivity mild). The test would have been repeated if the controls did not perform as anticipated and/or if both wells did not yield the same conclusion (e.g., one well passed and the other well failed).

Both the Non-buffered and the buffered Hypochlorous Acid Solutions showed no evidence of causing any cell lysis or toxicity. (See FIG. 9 and FIG. 10)

### Example 5

### Extended Stability Study

FIG. 11 shows the results of an extended stability study of electrochemically generated HOCl produced at targeted pH 6.9, stored in a HDPE jerry can, drum and tote and stored at room temperature. All batches were stabilized with blended of sodium phosphates and polyphosphates.

Every week samples of the HOCl stabilized solution were obtained from jerry can, drum and totes and analyzed for FAC, pH, ORP and Conductivity. Jerry can, drums and totes reopened once a week, closed after obtaining a sample and tested on a weekly basis. Comparison of the FAC and FAC of the all batches confirmed the stability of the stabilized (buffered) electrochemically HOCL with DIC.

The stability of 4 batches stabilized electrochemically generated HOCl is shown in FIG. 9. The ionic strength or solution salinity was not affected by the addition of DIC. The results demonstrate that a blend of sodium phosphate and polyphosphate as a stabilizer affects both the FAC and pH stability. Without being bound to any theory, in cases where the pH is above 5.5 and the buffering ability of DIC is minimal, the DIC acts as a stabilizer, in part, by scavenging free radicals generated by the dissociation of Hypochlorous Acid. The result is a minimal drop in FAC and pH over time.

Generally, it is assumed by NaOCl manufacturers that sodium hypochlorite solution loses approximately 20% of its titrable chlorine in the first 6 months and up to 60% within a year. One study determined that it would take 166 days for a solution of 25 mg/mL sodium hypochlorite solution at 20° C. to reach 20 mg/mL of free residual chlorine based on stability studies conducted at 50° C. and 70° C. and calculations with the Arrhenius Equation (See Nicoletti et al., “Shelf-Life of a 2.5% Sodium Hypochlorite Solution as Determined by Arrhenius Equation,” Braz Dent J (2009) 20(1): 27-31). Other studies have shown similar results (See “Product Characteristics, Sodium Hypochlorite-Stability PCH-1400-0007” PCH-1400-0007-W-EN (WW), Issue 1-May 2005, Published by Solvay Chemicals International SA).

Contrary to these assumptions, the buffered and non-buffered Hypochlorous Acid solution of the claimed invention retained greater than 80% of the initial level of titrable chlorine along with a pH shift of less than one unit over a period of six months.

Current “First Order Rate Equation” with 103113AX (20 weeks).

ln 250×10⁻⁶=−kt+ln 186×10⁻⁶

ln 250×10⁻⁶+kt+ln 186×10⁻⁶

−30.6993=−kt

−30.6993=−kt

At the time of sampling, hypochlorous acid solution was in the tote for 19 weeks. The goal of the stability testing is to prove 2 years or 104 weeks of shelf-life stability at ambient temperature. Therefore 20 is divided by 104 equaling 19%. We represent this value in the rate equation as t_{(20/104)} or 1/0.19.

−30.6993=−k×1/0.19−

30.6993×0.19=−k

5.833=k

Based on this equation, 1 week at 40 degrees Celsius equals 5.833 weeks.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A sanitizing solution having Hypochlorous Acid as active ingredient and a stabilizing amount of Dissolved Ionic Compounds (DIC), a Free Available Chlorine (FAC) content from about 10 to about 1000 parts per million, and a pH from about 4.0 to about 7.5.

2. The sanitizing solution of claim 1, wherein the DIC is selected from the group consisting of sodium phosphate, sodium polyphosphate, phosphate, polyphosphate of an alkali or an alkaline earth metal.

3. The sanitizing solution of claim 1, wherein said FAC content and said pH are stable for at least 12 months.

4. The sanitizing solution of claim 2 wherein said DIC is prepared by electrolysis of a diluted brine solution.

5. The sanitizing solution of claim 4, wherein the brine solution is a saturated brine solution and prepared in a brine tank wherein purified granular Sodium or Potassium Chloride is saturated in purified water and the saturated brine solution is pumped from the bottom of the brine tank and diluted with the purified water to achieve a predetermined conductivity in the electrolyte prior to electrolysis of the diluted brine solution.

6. The sanitizing solution of claim 5, wherein the targeted conductivity of the diluted brine solution is about 1:5 [200 ppm] compared to the FAC amount in the electrochemically generated Hypochlorous Acid.

7. The sanitizing solution of claim 5, wherein purified granular Sodium or Potassium Chloride is saturated in an electrochemically generated diluted Hydroxide solution to achieve and stabilize the pH of the saturated brine solution between 9 and 11.

8. The sanitizing solution of claim 1, wherein the stabilizing amount of DIC is typically, but not exclusively a sodium phosphate, sodium polyphosphate or a blended concentrated solution of orthophosphates, monophosphates, polyphosphates and other phosphates.

9. The sanitizing solution of claim 8, wherein the stabilizing amount of sodium phosphate, sodium polyphosphate or a blended DIC solution compared to the FAC amount in the electrochemically generated Hypochlorous Acid solution is about 1:250. [40 ppm].

10. The sanitizing solution of claim 9, wherein the amount of sodium phosphate, sodium polyphosphate or blended DIC solution compared to FAC amount the solution varies from about 1:100 to about 1:10,000 depending on concentration of the sodium phosphate, sodium polyphosphate or blended DIC solution [1 to 100 ppm].

11. The sanitizing solution of claim 2, wherein the sodium phosphate, sodium polyphosphate or blended DIC is contained within the electrochemically generated Hypochlorous Acid solution.

12. The sanitizing solution of claim 2, wherein the sodium phosphate, sodium polyphosphate or blended DIC solution is added to the electrochemically generated Hypochlorous Acid solution.

13. The sanitizing solution of claim 1, wherein the solution comprises Hypochlorous Acid produced by electrolysis of a saline solution, and the solution has an FAC content of from 10 to 1000 parts per million, a pH in the range of from 4 to 7.5, conductivity of from about 100 uS to about 15000 uS, and from 1 to 100 parts per million of sodium phosphate, sodium polyphosphate or blended DIC solution.

14. The sanitizing solution of claim 1, wherein the solution is formulated as a clear liquid, gel, cream, paste or foam.

15. A method for generating a sanitizing solution according to claim 1, comprising the steps of incorporating said DIC into said Hypochlorous Acid solution or a hydrogel formulation an in amount sufficient to stabilize the Hypochlorous Acid solution for at least 12 months.

16. A method for disinfecting or cleansing a mammalian tissue, comprising, applying said solution of claim 1 to a mammalian tissue.

17. The method of claim 16, wherein the mammalian tissue is infected.

18. The method of claim 17, wherein the tissue comprises a wound or burn.

19. The method of claim 17, wherein the sanitizing solution is applied to the affected area of a mammal having one or more dermatoses.

20. A method for disinfecting or sanitizing a hard surface by applying the solution of claim 1 to the hard surface.

21. A method of cleaning or sanitizing a food product by applying the solution of claim 1 directly or indirectly to the food product.

22. A method of disinfecting water by adding the solution of claim 1 to a source of water.

23. A method of sanitizing hands and tissue by applying the sanitizing solution of claim 1 directly on the skin.
